(19) United States
(12) Patent Application Publication
(10) Pub. No.: US 2026/0053451 A1
(43) Pub. Date: Feb. 26, 2026

(54) PATIENT-POSITIONING SYSTEM FOR USE IN MEDICAL TABLES

(71) Applicant: MAQUET GMBH, Rastatt (DE)

(72) Inventors: Jan Donat OLSZEWSKI, Rastatt (DE); Sebastian SCHMOLL, Rastatt (DE)

(21) Appl. No.: 19/102,825

(22) PCT Filed: Jul. 28, 2023
(86) PCT No.: PCT/EP2023/071043

§ 371 Date: Feb. 10, 2025

(30) Foreign Application Priority Data
Aug. 11, 2022 (DE) 10 2022 120 347.6

(51) Int. Cl. A61B 6/04

(52) U.S. Cl. CPC A61B 6/0407

(57) ABSTRACT

A patient positioning system (19), usable as a part of a medical table (10), wherein the patient positioning system (19) comprises: a patient bearing device (18) for bearing a patient, and a support device (17) on which the patient bearing device (18) lies, wherein the patient bearing device (18) is movable with regard to the support device (17) in order to move a patient thereon, wherein the support device (17) has several support rolls (40, 41) for bearing and displacing the patient bearing device (18), wherein the support rolls (40, 41) can be rotated about their respective rotational axis independently of one another, and wherein at least two of the support rolls (40, 41) form a first group and the support rolls (40, 41) of the first group can only be pivoted together with regard to other parts of the support device (17).

### TECHNICAL FIELD

The present disclosure relates to patient positioning systems for medical and in particular surgical tables. Further, the disclosure relates to medical tables with systems for patient positioning.

### BACKGROUND OF THE DISCLOSURE

During medical examinations or surgical procedures, patients can be borne on medical tables, in particular operating tables. Systems for patient positioning serve to bring patients into a desired position for medical examinations or procedures. Patient positioning systems can often be flexibly assembled and offer different possibilities of patient positioning.

Patient positioning systems can have exchangeable, detachably connectable segments. Often, some or all of the exchangeable segments are movable. Through the use of different exchangeable segments, a single medical table can be reconfigured in different ways for different patients and procedures.

During the examination or the procedure, patient usually lie or sit on a patient bearing device which is guided or borne via linear guides or on support rolls or via a combination of linear guides and support rolls and can be displaced in the longitudinal direction. In particular in the case of cantilevered or free-swinging systems, the patient bearing device can noticeably deform under load.

If only one support roll is used at a bearing site of the patient bearing device, the contact between the patient bearing device and the support roll remains maintained even in case of a deformation of the patient bearing device. However, if the load is supported by only one support roll, the surface pressure at the contact surface between the support roll and the patient bearing device is comparably high. With an elastomer coating on the support roll, the load is distributed to a larger surface, as the elastomer coating is flattened by the load at the contact surface. Still, often a larger support roll diameter has to be chosen in order to reach a contact surface which is as large as possible.

If two support rolls for bearing the patient bearing device are used at a bearing site of the patient bearing device, the load is distributed to both support rolls in case of a non-deformed patient bearing device. However, if the patient bearing device deforms under a load, the contact of the patient bearing device with one of the two support rolls can get lost so that the complete load lies on only one support roll.

### SUMMARY OF THE DISCLOSURE

It is an object of the present disclosure to create a patient positioning system with two or more support rolls for bearing the patient bearing device, wherein the load is distributed to as many support rolls as possible even in case of a deformation of the patient bearing device.

A patient positioning system according to a first aspect of the present disclosure can be used as a part of a medical table, in particular an operating table. The patient positioning system can have a patient bearing device for bearing a patient and a support device on which the patient bearing device lies. The patient bearing device can be movable with regard to the support device in order to move a patient thereon and position them for an examination or a surgical procedure.

In order to be able to move the patient bearing device on the support device, the support device contains several support rolls on which the patient bearing device is borne. The patient bearing device can be moved on the support rolls in a longitudinal direction or in a lateral direction, for example. The longitudinal direction, which can also be referred to as length direction, can correspond to the main direction or longitudinal axis of the patient bearing device. The lateral direction can extend perpendicular to the longitudinal direction.

The support rolls of the support device can be rotated about their respective rotational axis independently of one another. At least two of the support rolls form a first group. The support rolls of the first group can only be pivoted or tipped or trended together with regard to other parts of the support device.

By pivoting (or tipping or trending) the at least two support rolls together, the support rolls can automatically adapt to an underside of the patient bearing device if the patient bearing device is deformed. This causes all support rolls of the first group to be able to stay in contact with the patient bearing device, even if the patient bearing device bends due to a load. Consequently, the load can be distributed to the support rolls of the first group and does not have to be supported by only one of the support rolls. In particular, supporting forces can be distributed in a uniform manner to all support rolls of the first group.

The first group can comprise exactly two support rolls or more support rolls as well, for example three support rolls. If the first group has more than two support rolls, it can be useful to provide the support rolls and/or the underside of the patient bearing device with a flexible elastomer coating. If the surfaces of the support rolls and the underside of the patient bearing device are inflexible, the patient bearing device only lies on the two outer support rolls. The flexible coating can balance different distances between individual support rolls and the patient bearing device which can be caused by the curvature of the patient bearing device. Thus, a load distribution, even if it is uneven, on all support rolls can be achieved.

Compared to solutions with only one support roll, the contact surface with the patient bearing device can be increased even if a smaller support roll diameter is used.

In some designs, all support rolls of the support device can belong to the first group. In this case, the support device cannot contain any further support rolls apart from the support rolls of the first group. In other designs, the support device can contain further support rolls in addition to the support rolls of the first group. These support rolls can be assigned to a second group, for example. This is explained in detail below.

The support rolls can also be referred to as supporting rolls or carrier rolls.

In some designs, the patient bearing device can have an intermediate element which is borne on the support rolls and is in contact therewith. The other components of the patient bearing device can be detachably fastened on the intermediate element. In particular, the surface on which the patient is borne can be detachable from the intermediate element. However, the intermediate element is a part of the patient bearing device.

In some designs, the patient bearing device can be a surgical patient bearing device on which a patient is borne during a surgical procedure. The patient bearing device can be modularly formed and have a bearing surface main section which can be extended by coupling diverse bearing surface subsections. The bearing surface main section and the bearing surface subsections can have mechanical connection elements with which the bearing surface main and subsections can be detachably connected. Bearing surface subsections can be leg, foot or head sections, for example. Furthermore, the bearing surface subsections can also be extension or intermediate sections which are inserted between the bearing surface main section and the head section, for example. Further, the patient bearing device can serve to fasten accessory parts. Sliding or lateral rails can be attached on the sides of the bearing surface main and subsections. Accessory parts can be detachably fastened on the sliding or lateral rails. Furthermore, sliding or lateral rails can be provided at the head and/or foot end.

The patient positioning system can be provided for fastening on an operating table column of an operating table. The support device can have an intersection at its underside which serves to be fastened on an operating table column. The patient positioning system can be fixedly or detachably be connected to an operating table column. The operating table can be movable. A foot or a base of the operating table can have wheels or rolls with which the operating table can be moved on the ground. Alternatively, the foot or the base can be fixedly anchored on the ground.

In some designs, the patient positioning system can be designed so that it can be detachably connected to a movable surgical patient transporter and a column of an operating table. The patient positioning system can be mounted on the patient transporter before a surgical procedure. The patient can be brought to the operating table with the patient transporter. There, the patient positioning system can be fastened to the operating table column and be decoupled from the patient transporter. A large part of the preparations to the surgical procedure can be done while the patient positioning system is mounted on the patient transporter. For example, the patient bearing device can be assembled from individual segments and accessory parts and the patient can be prepared for the surgical procedure. Thus, it is possible to fasten the patient positioning system on the operating table column after completion of the preparations.

In some designs, linear guides in combination with the support rolls can be used in order to be able to displace the patient bearing device in the longitudinal or lateral direction.

In some designs, the at least two support rolls of the first group can be aligned substantially parallel to one another and furthermore, the at least two support rolls can be pivotably connected to the other parts of the support device by one or more pivoting engagements. In particular, the at least two support rolls can be connected to one another by at least a concave and/or convex engagement, at least an arcuate engagement, at least an elastic connection, at least a borehole connection, at least a pivotable bearing block, at least a hinge or at least an axis connection.

In some designs, the support rolls can extend below the patient bearing device along the total width of the patient bearing device or also beyond. The width of the patient bearing device runs perpendicular to the longitudinal direction of the patient bearing device along which the support of a patient is provided. With the width of the support rolls corresponding to the width of the patient bearing device, an improved bearing can be achieved.

In some designs, the patient positioning system can have bearing blocks which each bear ends of the at least two support rolls of the first group so that the rotational axes of the at least two support rolls are aligned substantially parallel to one another. The bearing blocks can be positioned on the two ends of the at least two support rolls. The at least two support rolls of the first group can be borne in two bearing blocks in total. The bearing blocks can have recesses in which the ends of the at least two support rolls are borne. Due to the bearing in the bearing blocks, the rotational axes of the at least two support rolls can be aligned parallel to one another and have a fixed distance. The bearing blocks enable that the at least two support rolls of the first group can rotate about their respective rotational axis independently of one another, but can only pivot together with regard to other parts of the support device.

As soon as the patient bearing device is in contact with the at least two support rolls, the support rolls align and stabilise one another. Due to the use of at least two support rolls, the load is distributed to several support rolls, whereby the forces on the bearings with which the support rolls are borne in the bearing blocks are also reduced.

In some designs, the two bearing blocks which support the at least two support rolls of the first group can be pivotable about a common pivot axis. By pivoting the bearing blocks, the at least two support rolls are pivoted as well. Pivoting the bearing blocks enables the at least two support rolls of the first group to automatically adapt to the underside of the patient bearing device if the patient bearing device is deformed.

In some designs, the bearing blocks can be rotatably borne in order to thereby be pivotable with regard to other parts of the support device.

In some designs, the patient positioning system can have support blocks which serve the pivotable bearing of the bearing blocks. In particular, one support block can be provided for every bearing block. A bearing block can be borne on the respective support block so that the bearing block can be pivoted with regard to the support block and in particular further parts of the support device. Each of the support blocks can have a first bearing section on which a second bearing section of the respective bearing block is borne. The first bearing sections of the support blocks can be formed to be complementary to the second bearing sections of the bearing blocks. This enables a pivoting of a bearing section with regard to the complementary, other bearing section in an improved manner.

The bearing blocks can slide on the support blocks and thus pivot or tip or trend. A coupling between the two bearing blocks in which the at least two support rolls of the first group are borne is not required for a synchronous pivoting and does not have to be provided either.

The bearing blocks can be connected to the support blocks in a non-fixed, in particular loose manner. The bearing blocks can simply sit on the support blocks. The bearing blocks are pressed against the support blocks by their own weight, the weight of the support rolls and the weight of the patient bearing device lying above.

In some designs, the support blocks can each have a bore in which a threaded screw is guided with which the respective support block is fastened on a base element of the support device. The threaded screws can be provided for height adjustment of the support blocks.

In some designs, the first bearing sections of the support blocks can engage the second bearing sections of the bearing blocks.

In some designs, the first bearing sections of the support blocks can each be formed to be concave and the second bearing sections of the bearing blocks can each be formed to be convex. Alternatively, the first bearing sections of the support blocks can each be formed to be convex and the second bearing sections of the bearing blocks can each be formed to be concave. The complementary and in particular convex/concave formation of the contact surface between the support blocks and the bearing blocks enables the pivoting of the bearing blocks with regard to the support blocks.

In some designs, the first bearing sections and the second bearing sections can each have an arcuate surface. The associated centre point angle or central angle of the arc can be 180 degrees, for example. The centre point angle or central angle of the arc is the angle whose apex is in the centre of the circle and whose legs intersect the limitation points of the arc. In case of a centre point angle of 180 degrees, the first bearing sections of the support blocks can be semicircular. However, the centre point angle can also have another suitable value. Further, the centre point angles of the arcuate surfaces of the first bearing sections of the support blocks can be different from the centre point angles of the arcuate surfaces of the second bearing sections of the bearing blocks.

In some designs, the at least two support rolls of the first group can be adjusted in height. In particular, the support blocks can be adjusted in height, whereby the height of the at least two support rolls can be changed. For example, the distance of the support blocks from a surface area of the support device can be adjusted by means of threaded screws.

In some designs, at least two of the support rolls can form a second group, which means that the support device has at least four support rolls in total, wherein two respective support rolls are assigned to the first or second group. The support rolls of the first group and of the second group can each be borne in two bearing blocks and the bearing blocks can be pivotably borne on support blocks. The support rolls of the second group can be pivoted together with regard to other parts of the support device just like the support rolls of the first group. Further, the support blocks can be fastened on a support element which, for its part, is pivotably borne. The pivotable support element guarantees that all support rolls keep contact with the patient bearing device in case the patient bearing device is deformed. The support element can be a fixed plate, for example, on the upper side of which the support blocks are mounted. The pivotable bearing of the support element can be achieved by pivotably bearing the support element on further support blocks. The support rolls and bearing blocks of the first and the second group of these designs can have the designs described in the present disclosure.

The extension of the positioning system to the second group with at least two support rolls which can be pivoted together and the pivotable support element has several advantages. On the one hand, the load is not distributed to at least two support rolls, but to at least four support rolls. On the other hand, the support rolls can automatically adapt to the underside of the patient bearing device even if the patient bearing device is strongly deformed. Despite the curvature, all support rolls of the support device can stay in contact with the patient bearing device, whereby the load is furthermore distributed to all support rolls.

In some designs, at least two of the support rolls can form a second group. The support rolls of the first group and of the second group can each be borne in two bearing blocks and the bearing blocks can be pivotably borne on support blocks. The support rolls of the first group and of the second group can each be borne in two bearing blocks and the bearing blocks can be pivotably borne on support blocks. The support rolls and bearing blocks of the first and the second group of these designs can have the designs described in the present disclosure. Further, the support blocks which support the support rolls of the first or the second group can be spaced apart from one another so that an end of the patient bearing device is borne on the support rolls of the first group and the other end of the patient bearing device is borne on the support rolls of the second group. Consequently, the support rolls are not used for one-sided bearing, such as with cantilevered or free-swinging systems, but for two-sided bearing of the patient bearing device. The distance of the support blocks of the first group to the support blocks of the second group can in particular be at least 150 cm or 200 cm. In these designs as well, the support rolls of the first and the second group always adapt to the bending of the patient bearing device due to their pivotable bearing. Furthermore, the number of the support rolls on each end of the patient bearing device can be increased by at least one respective further group of support rolls which can be pivoted together, wherein the groups of support rolls on each end can be arranged on a pivotable support element as described above so that all support rolls can stay in contact with the patient bearing device in case of a deformation of the patient bearing device.

In some designs, support blocks on which the bearing blocks sit in a pivotable manner can be dispensed with and the bearing blocks can instead be mounted via a bore, for example. The bearing blocks can each have a recess, for example, in particular a bore, which is engaged by a respective wave-shaped element for pivotable bearing of the bearing blocks. Alternatively, the bearing blocks can each have a wave-shaped element which is rotatably borne for pivotable bearing of the bearing blocks. The wave-shaped element can be a support, for example, with a cylinder-shaped section which is either fastened on the support device or on the bearing blocks and engages the recess in the bearing blocks or sits on a bearing element of the support device.

In some designs, one or more or all support rolls can each have a supporting section for bearing the patient bearing device and a bearing section for bearing the respective support roll which is fixedly connected to the supporting section. The bearing section can be rotatably connected to the bearing blocks by means of needle bearings, for example. Instead of needle bearings, a plurality of other bearings, in particular rolling or sliding bearings, can be used.

In some designs, the supporting sections of one or more or all support rolls for bearing the patient bearing device can be coated with a flexible elastomer, whereby a larger contact surface with the patient bearing device is achieved. Alternatively or additionally, the underside of the patient bearing device, which lies on the support rolls, can be coated with a flexible elastomer. As a further alternative, it can be provided that the support rolls or at least some of the support rolls and/or the underside of the patient bearing device are not coated with an elastomer.

Apart from a flat underside, the underside of the patient bearing device can also be curved about the longitudinal axis of the patient bearing device or can have a profile with another shape. The supporting sections of one or more or all support rolls can have profiles which are shaped so that they receive the profile of the underside of the patient bearing device. In other words, the profile of one or more or all support rolls can be complementary to the profile of the underside of the patient bearing device. This causes an enlarged contact surface between the patient bearing device and the support rolls.

In some designs, the patient bearing device can be displaceable in the longitudinal direction with regard to the support device. In particular, the patient bearing device can be rollable over the at least two support rolls of the first group during the displacement in the longitudinal direction. If the support device has further support rolls, the patient bearing device can also be rolled over these support rolls.

In some designs, the patient bearing device can comprise a longitudinal and substantially flat surface for bearing a patient thereon, which is substantially radiotransparent. The patient positioning system can be used as a part of a medical imaging table.

In some designs, at least a part of the patient bearing device can be elongated and substantially planar or flat. The patient bearing device can be able to bend under a patient load. If the patient bearing device bends, the at least two support rolls of the first group can pivot together with regard to the other parts of the support device so that the at least two support rolls stay in contact with the patient bearing device during the bending.

In some designs, the at least two support rolls of the first group can be pivotable together only in a limited pivot range, in particular in a pivot range of at most 20 degrees or 15 degrees or 10 degrees or less, with regard to the other parts of the support device.

In some designs, the at least two support rolls of the first group can be, in particular in general, be parallel to one another and the at least two support rolls can pivot together with at least a part of a common support device; and wherein the support device has a curved, concave section on a bottom surface, wherein a bent, upwardly convex support block is arranged below the support device and supports the support device, wherein in particular the support block is semicircular; and wherein the support device lies on the support block and is displaceable on the support block for pivoting the at least two support rolls.

According to a second aspect of the present disclosure, a medical table, in particular an operating table, is provided, which has a column and a patient positioning system according to the first aspect. The patient positioning system can be fixedly mounted on the column or alternatively be detachably connectable to the column.

The medical table according to the second aspect can have all designs which are described in the present disclosure in connection with the patient positioning system according to the first aspect.

The present disclosure also comprises circuits and/or electronic instructions for controlling medical tables and remote controls, displays and user interfaces for use with medical tables.

### SHORT DESCRIPTION OF THE DRAWINGS

Example embodiments of the present disclosure are explained in more detail hereinafter with the figures: In the drawings:

FIGS. 1A and 1B show schematic lateral views of an operating table with a patient positioned on a patient bearing device of the operating table;

FIG. 2 shows a schematic representation of a patient positioning system according to the disclosure;

FIG. 3 shows a schematic representation of a bearing block borne on a support block;

FIG. 4 shows a schematic representation of a support device;

FIG. 5 shows a schematic representation of a patient positioning system according to the disclosure with a support device and a patient bearing device;

FIGS. 6A to 6D show schematic representations of fixedly anchored support rolls and pivotably borne support rolls;

FIG. 7 shows a schematic representation of a patient positioning system according to the disclosure with two groups of pivotably borne support rolls;

FIG. 8 shows a schematic representation of a patient positioning system according to the disclosure with a patient bearing device borne on both ends on support rolls;

FIG. 9 shows a schematic representation of a bearing block with a wave-shaped element for pivotable bearing of the bearing block; and

FIG. 10 shows a schematic representation of a patient bearing device with a profiled underside and a support roll with a profiled supporting section.

### DETAILED DESCRIPTION OF THE FIGURES

In the following description, example embodiments of the present disclosure are described with reference to the drawings. The drawings are not necessarily drawn to scale, but are intended to only schematically illustrate the respective features.

It is to be noted that the features and components described below can each be combined with one another, irrespective of whether they were described in connection with a single embodiment. The combination of features in the respective embodiments only serves to illustrate the general setup and the functioning of the claimed device.

In the figures, identical or similar elements are indicated with identical reference numerals as far as this is appropriate.

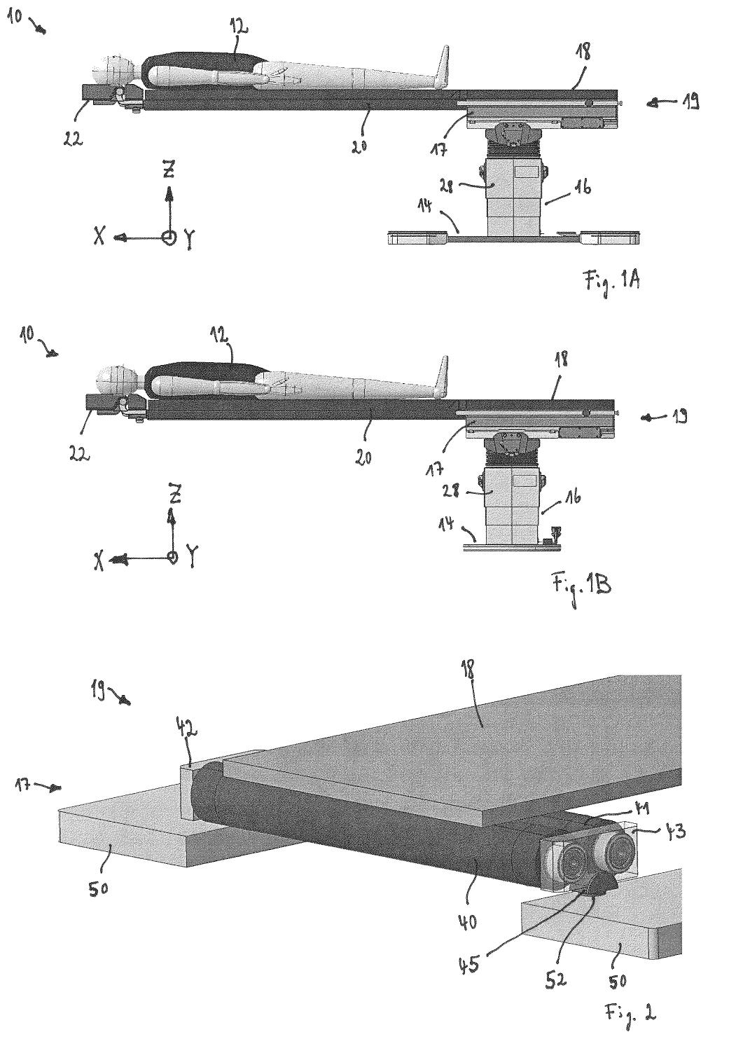

FIGS. 1A and 1B schematically show a mobile operating table 10 which can be used for bearing a patient 12 during a surgical procedure and for transporting them. The mobile operating table 10 comprises, from bottom to top, a foot 14 for placing the operating table 10 on an underground, an operating table column 16 connected to the foot 14, a support device 17 fastened on an upper end of the operating table column 16 and a patient bearing device 18 which lies on the support device 17. The support device 17 and the patient bearing device 18 together form a patient positioning system 19. The patient positioning system 19 can be firmly connected to the operating table column 16 or alternatively be detachably fastened on the operating table column 16.

The patient bearing device 18 is formed modularly and serves to bear the patient 12. The patient bearing device 18 comprises a bearing surface main section 20 which can be extended as desired by coupling diverse bearing surface subsections. In FIGS. 1A and 1B, a head section 22 is coupled to the bearing surface main section 20 as a bearing surface subsection.

Depending on the type of surgical procedure to be performed, the patient bearing device 18 of the operating table 10 can be brought to a suitable height and both tilted and trended. Further, the patient bearing device 18 can be adjusted in the longitudinal direction.

The operating table column 16 is formed to be adjustable in height and has an internal mechanics for adjusting the height of the patient bearing device 18. The mechanics is arranged in a housing 28 which protects the components from pollution.

FIG. 1A shows the operating table 10 in a mobile design. The foot 14 has wheels or rolls with which the operating table 10 can be moved on the ground. Alternatively, the foot 14 can be fixedly anchored on the ground, as is shown in FIG. 1B.

For better illustration, a Cartesian coordinate system X-Y-Z is drawn in FIGS. 1A and 1B. The X-axis and the Y-axis are horizontal axes, the Z-axis is a perpendicular axis. The X-axis indicates the longitudinal direction or length direction and the Y-axis indicates the lateral direction.

FIG. 2 schematically shows a cutout of a patient positioning system 19 according to the disclosure. The patient positioning system 19 is a system according to the first aspect of the present disclosure and can be mounted on a column of a medical table according to the second aspect.

The patient positioning system 19 contains a support device 17 on which a patient bearing device 18 lies. The patient bearing device 18 comprises a surface on which a patient can be borne and is movable in the longitudinal direction with regard to the support device 17.

The support device 17 contains support rolls 40, 41 which together form a first group of support rolls. The ends of the support rolls 40, 41 are each borne in recesses of bearing blocks 42, 43 with needle bearings. Through the bearing blocks 42, 43, the rotational axes of the support rolls 40, 41 are aligned parallel to one another and the support rolls 40, 41 can be rotated about their respective rotational axis independently of one another.

The bearing blocks 42, 43 are each borne on a support block 45. In FIG. 2, only one support block 45 can be recognised, the other support block 45 is covered by the support rolls 40, 41.

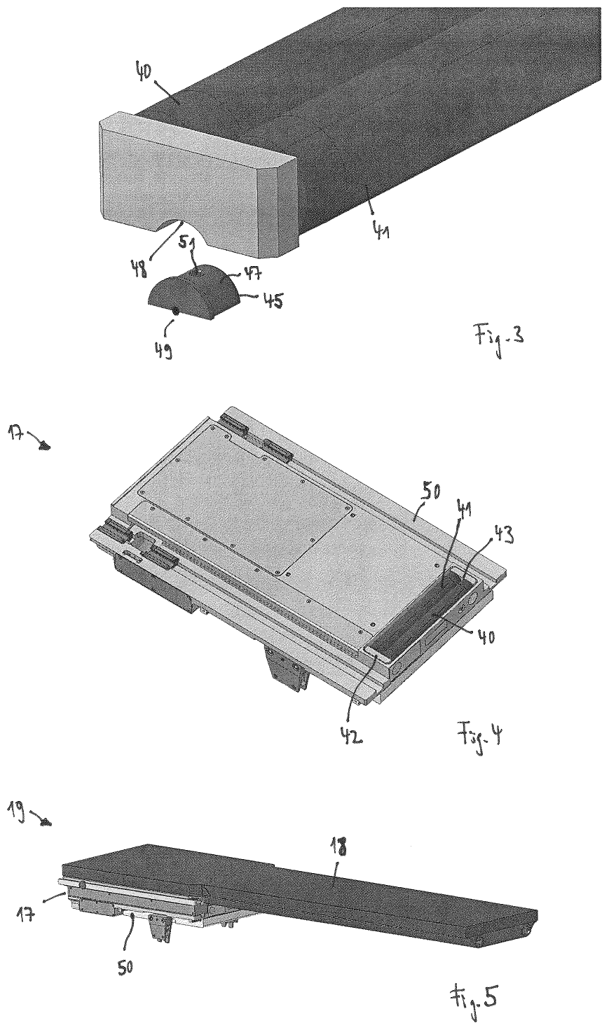

FIG. 3 shows details of the bearing block 43 and of the support block 45. The other bearing and support blocks 42, 45, not visible in FIG. 3, are designed in the same way. The support blocks 45 each have a first bearing section 47 on their upper side on which a second bearing section 48 of the respective bearing block 42, 43 is borne. The first and second bearing sections 47, 48 are formed complementarily in order to enable a pivoting of the respective bearing block 42, 43 on the support blocks 45.

The bearing blocks 45 represented in FIGS. 2 and 3 have a convexly formed first bearing section 47 which is semicircular in cross-section. Correspondingly, the second bearing section 48 of the bearing blocks 42, 43 is formed concavely and the cross-section of the second bearing section 48 is arcuate.

The bearing of the bearing blocks 42, 43 on the support blocks 45 enables to pivot the bearing blocks 42, 43 with regard to the respective support block 45. The axis about which the pivot movement is performed corresponds to a centre axis 49 of the semicircular support blocks 45 which is drawn in FIG. 3 for better understanding.

Since the support rolls 40, 41 are guided in the bearing blocks 42, 43, a pivoting of the bearing blocks 42, 43 causes a common pivoting of the support rolls 40, 41 with regard to the support blocks 45 and in particular a base element 50 of the support device 17, represented in FIG. 2, on which the support blocks 45 are fastened.

The support blocks 45 each have a bore 51 in which a threaded screw 52 is guided, with which the support blocks 45 are fastened on the base element 50. The threaded screws 52 enable to adjust the height of the support blocks 45.

If the patient bearing device 18 is placed on the support rolls 40, 41, the support rolls 40, 41 align and stabilise one another. Further, the load is distributed to both support rolls 40, 41. In case of a displacement of the patient bearing device 18 in the longitudinal direction, the support rolls 40, 41 rotate about their respective rotational axis.

By pivoting the support rolls 40, 41 together, the support rolls 40, 41 automatically adapt to the underside of the patient bearing device 18 even if the patient bearing device 18 is deformed.

FIG. 4, in perspective, shows the support device 17 with the base element 50 and the support rolls 40, 41 as well as the bearing blocks 42, 43 which are inserted into a recess of the base element 50 so that support rolls 40, 41 protrude beyond the upper side of the base element 50 so that the patient bearing device 18 can be moved on the support rolls 40, 41.

FIG. 5 shows the patient positioning system 19 with the support device 17 and the patient bearing device 18 borne on the support device 17.

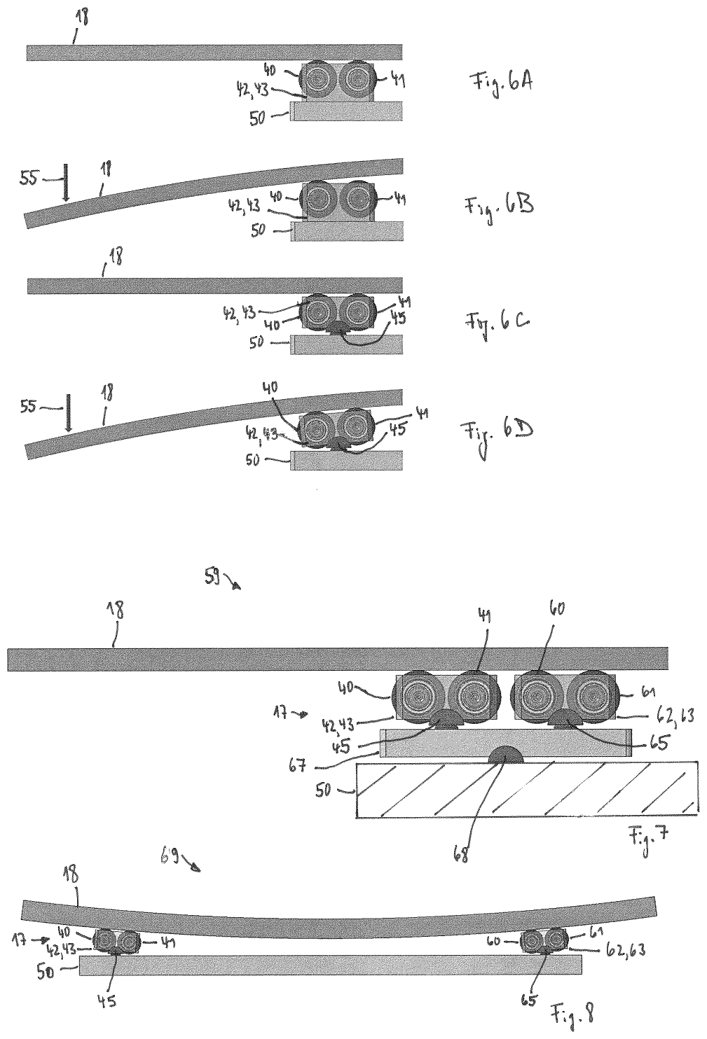

FIGS. 6A to 6D illustrate differences between pivotably borne bearing blocks 42, 43 and bearing blocks 42, 43 fixedly connected to the base element 50.

In FIGS. 6A and 6B, the bearing blocks 42, 43 are rigidly fastened on the base element 50 of the support device 17, while in FIGS. 6C and 6D, the bearing blocks 42, 43 are pivotably borne on the support blocks 45, as was described further above in connection with FIGS. 2 to 5.

In FIGS. 6A and 6C, no additional load acts on the patient bearing device 18. In FIGS. 6B and 6D, an additional load, which can be caused by a patient, for example, acts on the patient bearing device 18. The additional load is marked in FIGS. 6B and 6D with an arrow 55.

FIGS. 6A and 6C show that without an additional load, the support rolls 40, 41 of the two designs are in contact with the underside of the patient bearing device 18 and the load caused by the patient bearing device 18 is distributed to both support rolls 40, 41.

However, if the additional load symbolised by the arrow 55 acts on the patient bearing device 18, this load, with the design with the fixedly mounted bearing blocks 42, 43 and the thus not pivotable support rolls 40, 41 causes them not to be in contact with the support roll 41 anymore due to the deformation of the patient bearing device 18, as FIG. 6B shows. In this case, the load fully acts on the support roll 40.

In contrast, both support rolls 40, 41 stay in contact with the underside of the patient bearing device 18 even in case of a deformation of the patient bearing device 18 if the support rolls 40, 41 can be pivoted together, as FIG. 6D shows. By pivoting the support rolls 40, 41 together, the support rolls 40, 41 can automatically adapt to the underside of the patient bearing device 18 if the patient bearing device 18 is deformed. In this case, the load is distributed to both support rolls 40, 41 and does not have to be supported by the support roll 40 alone.

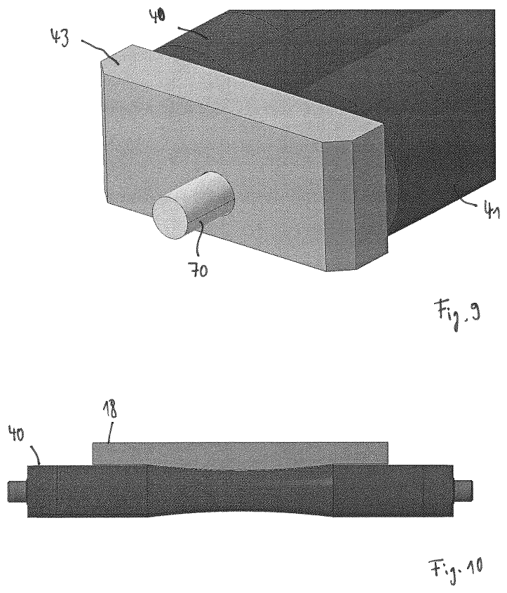

FIG. 7 schematically shows a patient positioning system 59 which represents a further development of the patient positioning system 19 described above. Apart from the support rolls 40, 41 of the first group of support rolls, the support device 17 contains a second group of support rolls 60, 61 which are borne in respective bearing blocks 62, 63 on their ends. The support rolls 60, 61 and the bearing blocks 62, 63 are constructed in the same way as the support rolls 40, 41 or the bearing blocks 42, 43. Further, the bearing blocks 62, 63 are pivotably borne on support blocks 65 which are constructed in the same way as the support blocks 45. Therefore, the bearing blocks 62, 63 can be pivoted together with the support rolls 60, 61 with regard to the support blocks 65.

In FIG. 7, the support blocks 45, 65 are not directly mounted onto the base element 50 of the support device 50, but are fastened on a support element 67. The support element 67, on the other hand, is pivotably borne on two support blocks 68 which are fastened to the base element 50. The support blocks 68 are formed in the same or a similar manner as the support blocks 45, 65. Therefore, the support element 67 can pivot with regard to the base element 50.

In the design represented in FIG. 7, the load is distributed to the four support rolls 40, 41, 60, 61. The pivotable bearing of both the support rolls 40, 41, 60, 61 and of the support element 67 guarantees that all support rolls 40, 41, 60, 61 automatically adapt to the underside of the patient bearing device 18 if the patient bearing device 18 is deformed.

FIG. 8 schematically shows a patient positioning system 69 which is a further development of the patient positioning system 19 described above. As in FIG. 7, the support device 17 in FIG. 8 comprises a first support roll group with the support rolls 40, 41 and a second support roll group with the support rolls 60, 61 which are borne in respective bearing blocks 42, 43, 62, 63 on their ends. Further, the bearing blocks 42, 43, 62, 63 are pivotably borne on support blocks 45 or 65. In FIG. 8, the support blocks 45, 65 are directly fastened on the base element 50 of the support device 17 so that the bearing blocks 42, 43, 62, 63 are pivotable together with the respective support rolls 40, 41, 60, 61 with regard to the base element 50.

Furthermore, the support blocks 45, 65 are spaced apart from one another so that an end of the patient bearing device 18 is borne on the support rolls 40, 41 of the first group and the other end of the patient bearing device is borne on the support rolls 60, 61 of the second group. Consequently, the support rolls 40, 41, 60, 61 are used for bearing the patient bearing device 18 on both sides.

FIG. 9 schematically shows a further design of the bearing blocks. The bearing block 43 represented in FIG. 9 does not sit on a bearing block, but has a wave-shaped element 70 which protrudes on the side of the bearing block 43 facing away from the support rolls 40, 41 and which is aligned parallel to the rotational axes of the support rolls 40, 41. The wave-shaped element 70 is rotatably borne in order to enable a pivoting of the bearing block 43 and of the support rolls 40, 41.

FIG. 10 shows the patient bearing device 18 with a profiled underside. A supporting section of the support roll 40, which is provided for bearing the patient bearing device 18, also has a profiling in sections which is complementary to the profiling of the patient bearing device 18. Thus, a larger contact surface between the patient bearing device 18 and the support roll 40 is achieved. The other support rolls 41, 60, 61 can be designed in the same way.

Example embodiments and variants corresponding to the present disclosure are described in the following list of clauses and options:
Clause 1: A patient positioning system (19, 59, 69) usable as a part of a medical table (10), wherein the patient positioning system (19, 59, 69) comprises:
   a patient bearing device (18) for bearing a patient, and
   a support device (17) on which the patient bearing device (18) lies,
   wherein the patient bearing device (18) is movable with regard to the support device (17) in order to move a patient thereon,
   wherein the support device (17) has several support rolls (40, 41) for bearing and displacing the patient bearing device (18),
   wherein the support rolls (40, 41) can be rotated about their respective rotational axis independently of one another, and
   wherein at least two of the support rolls (40, 41) form a first group and the support rolls (40, 41) of the first group can be pivoted only together with regard to other parts of the support device (17).
Clause 2: The patient positioning system (19, 59, 69) according to clause 1, wherein the at least two support rolls (40, 41) of the first group are aligned substantially parallel to one another and the at least two support rolls (40, 41) are pivotably connected to the other parts of the support device (17) by one or more pivoting engagements, in particular by at least one concave and/or convex engagement, an arcuate engagement, an elastic connection, a borehole connection, a pivotable bearing block, a hinge or an axis connection.
Clause 3: The patient positioning system (19, 59, 69) according to clause 1 or 2, further comprising bearing blocks (42, 43) which each bear ends of the at least two support rolls (40, 41) of the first group so that the rotational axes of the at least two support rolls (40, 41) are aligned substantially parallel to one another.
Clause 4: The patient positioning system (19, 59, 69) according to clause 3, wherein the bearing blocks (42, 43) can be pivoted about a common pivot axis.
Clause 5: The patient positioning system (19, 59, 69) according to clause 3 or 4, further comprising support blocks (45) for pivotably bearing the bearing blocks (42, 43), wherein the support blocks (45) each have a first bearing section (47) on which a respective second bearing section (48) of the bearing blocks (42, 43) is borne, and wherein the first bearing sections (47) are formed complementarily to the second bearing sections (48).
Clause 6: The patient positioning system (19, 59, 69) according to clause 5,
   wherein the first bearing sections (47) are each formed to be concave and the second bearing sections (48) are each formed to be convex, or
   wherein the first bearing sections (47) are each formed to be convex and the second bearing sections (48) are each formed to be concave.
Clause 7: The patient positioning system (19, 59, 69) according to clause 5 or 6,
   wherein the first bearing sections (47) and the second bearing sections (48) each have an arcuate surface.
Clause 8: The patient positioning system (19, 59, 69) according to any one of the preceding clauses, wherein the at least two of the support rolls (40, 41) of the first group are adjustable in height.
Clause 9: The patient positioning system (59) according to any one of the preceding clauses,
   wherein at least two of the support rolls (60, 61) form a second group,
   wherein the support rolls (40, 41, 60, 61) of the first group and of the second group are each borne in two bearing blocks (42, 43, 62, 63),
   wherein the bearing blocks (42, 43, 62, 63) are pivotably borne on support blocks (45, 65), and
   wherein the support blocks (45, 65) are fastened on a support element (67) which is pivotably borne.
Clause 10: The patient positioning system (69) according to any one of the preceding clauses,
   wherein at least two of the support rolls (60, 61) form a second group,
   wherein the support rolls (40, 41, 60, 61) of the first group and of the second group are each borne in two bearing blocks (42, 43, 62, 63),
   wherein the bearing blocks (42, 43, 62, 63) are pivotably borne on support blocks (45, 65), and
   wherein the support blocks (45, 65) are spaced apart from one another so that an end of the patient bearing device (18) can be borne on the at least two support rolls (40, 41) of the first group and the other end of the patient bearing device (18) can be borne on the at least two support rolls (60, 61) of the second group.
Clause 11: The patient positioning system (19, 59, 69) according to clause 3 or 4,
   wherein the bearing blocks (42, 43) each have a recess which is engaged by a respective wave-shaped element for pivotable bearing of the bearing blocks (42, 43), or
   wherein the bearing blocks (42, 43) each have a wave-shaped element (70) which is rotatably borne for pivotable bearing of the bearing blocks (42, 43).
Clause 12: The patient positioning system (19, 59, 69) according to any one of claims 3 to 10,
   wherein at least one of the support rolls (40, 41, 60, 61) has a supporting section for bearing the patient bearing device (18) and a bearing section for bearing the support roll (40, 41, 60, 61) which is fixedly connected to the supporting section, and
   wherein the bearing section is rotatably connected to the bearing blocks (42, 43) via bearings, in particular rolling, sliding or needle bearings.
Clause 13: The patient positioning system (19, 59, 69) according to any one of the preceding clauses,
   wherein at least one of the support rolls (40, 41, 60, 61) has a supporting section for bearing an underside of the patient bearing device (18), and
   wherein the supporting section and/or the underside of the patient bearing device (18) are coated with an elastomer.
Clause 14: the patient positioning system (19, 59, 69) according to any one of the preceding clauses,
   wherein at least one of the support rolls (40, 41, 60, 61) has a supporting section for bearing the patient bearing device (18), and
   wherein the supporting section has a flat or profiled surface.
Clause 15: The patient positioning system (19, 59, 69) according to any one of the preceding clauses,
   wherein at least one of the support rolls (40, 41, 60, 61) has a supporting section for bearing the patient bearing device (18), and
   wherein the patient bearing device (18) has a surface for bearing on the supporting section which is formed complementary to the surface of the supporting section.
Clause 16: The patient positioning system (19, 59, 69) according to any one of the preceding clauses, wherein the patient bearing device (18) can be displaced in the longitudinal direction with regard to the support device (17), wherein in particular the patient bearing device (18) can be rolled over the at least two support rolls (40, 41) of the first group during the displacement in the longitudinal direction.
Clause 17: the patient positioning system (19, 59, 69) according to any one of the preceding clauses,
   wherein the patient bearing device (18) comprises a longitudinal and substantially flat surface for bearing a patient thereon, which is substantially radiotransparent, and
   wherein the patient positioning system (19, 59, 69) can be used as a part of a medical imaging table.
Clause 18: The patient positioning system (19, 59, 69) according to any one of the preceding clauses,
   wherein at least a part of the patient bearing device (18) is elongated and substantially planar,
   wherein the patient bearing device (18) is able to bend under a patient load, and
   wherein, if the patient bearing device (18) bends, the at least two support rolls (40, 41) of the first group pivot together with regard to the other parts of the support device (17) so that the at least two support rolls (40, 41) stay in contact with the patient bearing device (18) during the bending.
Clause 19: The patient positioning system (19, 59, 69) according to any one of the preceding clauses, wherein the at least two support rolls (40, 41) of the first group can be pivoted together only in a limited pivot range, in particular in a pivot range of 20 degrees or less, with regard to the other parts of the support device (17).
Clause 20: The patient positioning system (19, 59, 69) according to any one of the preceding clauses, wherein the at least two support rolls (40, 41) of the first group are generally parallel to one another and the at least two support rolls (40, 41) pivot together with at least a part of a common support device (17); and
   wherein the support device (17) has a curved, concave section on a bottom surface,
   wherein a bent, upwardly convex support block (45) is arranged below the support device (17) and supports the support device (17), wherein in particular the support block (45) is semicircular; and
   wherein the support device (17) lies on the support block (45) and can be displaced on the support block (45) for pivoting the at least two support rolls (40, 41).
Clause 21: A medical table (10) with a column and a patient positioning system (19, 59, 69) according to any one of the preceding clauses, wherein the patient positioning system (19, 59, 69) is fixedly mounted on the column or can be detachably connected to the column.

1.-21. (canceled)

22. A patient positioning system usable as a part of a medical table, the patient positioning system comprising:
a patient bearing device configured to bear a patient; and
a support device on which the patient bearing device lies,
the patient bearing device being movable with respect to the support device to thereby facilitate movement of a patient thereon,
the support device comprising a plurality of support rolls configured to bear and displace the patient bearing device,
the support rolls being rotatable about their respective rotational axes independently of one another, and
at least two of the support rolls form a first group and the at least two support rolls of the first group are adjustable in height and can only be pivoted together with respect to other parts of the support device.

23. The patient positioning system according to claim 22, wherein the at least two support rolls of the first group are aligned substantially parallel to one another, and the at least two support rolls of the first group are pivotably connected to the other parts of the support device by one or more pivoting engagements, and/or the at least two support rolls of the first group extend below the patient bearing device along the total width of the patient bearing device.

24. The patient positioning system according to claim 22, further comprising first and second bearing blocks, the first bearing block bearing first ends of the at least two support rolls of the first group and the second bearing block bearing second ends of the at least two support rolls of the first group such that the rotational axes of the at least two support rolls of the first group are aligned substantially parallel to one another.

25. The patient positioning system according to claim 24, wherein the first and second bearing blocks can be pivoted about a common pivot axis.

26. The patient positioning system according to claim 24, further comprising first and second support blocks configured to pivotably bear the first and second bearing blocks, wherein the first and second support blocks each have a first bearing section on which a respective second bearing section of the corresponding first or second bearing block is received, each of the first bearing sections of the first and second support blocks having a shape that is complementary to a shape of the second bearing section of the corresponding first or second bearing block.

27. The patient positioning system according to claim 26, wherein:
the first bearing sections of the first and second support blocks are each concave, and the second bearing sections of the first and second bearing blocks are each convex, or
the first bearing sections of the first and second support blocks are each convex, and the second bearing sections of the first and second bearing blocks are each concave.

28. The patient positioning system according to claim 22, wherein the first and second support blocks each have a bore configured to receive a threaded element configured to adjustably connect the respective first or second support block to a base element of the support device and to adjust the height of the at least two support rolls of the first group.

29. The patient positioning system according to claim 24, wherein:
at least one of the at least two support rolls of the first group has a supporting section configured to bear the patient bearing device, and a bearing section configured to rotatably connect the at least one support roll to its respective one of the first and second bearing blocks, the bearing section being fixedly connected to the supporting section, and
the bearing section is rotatably connected to the first and second bearing blocks via bearings.

30. The patient positioning system according to claim 22, wherein:
at least one of the at least two support rolls of the first group has a supporting section configured to bear an underside of the patient bearing device, and
the supporting section and/or the underside of the patient bearing device are coated with an elastomer.

31. The patient positioning system according to claim 22, wherein:
at least one of the at least two support rolls of the first group has a supporting section configured to bear the patient bearing device, and
the supporting section has a flat or profiled surface.

32. The patient positioning system according to claim 22, wherein:
at least one of the at least two support rolls of the first group has a supporting section configured to bear the patient bearing device, and
the patient bearing device has a surface configured to be borne by the supporting section, the surface of the patient bearing device having a shape that is complementary to a shape of the supporting section.

33. The patient positioning system according to claim 22, wherein:
the patient bearing device comprises a longitudinal and substantially flat surface that is substantially radiotransparent and configured to bear a patient thereon, and
the patient positioning system is configured for use as a part of a medical imaging table.

34. The patient positioning system according to claim 22, wherein:
at least a part of the patient bearing device is elongated and substantially planar,
the patient bearing device is bendable under a patient load, and
when the patient bearing device bends, the at least two support rolls of the first group pivot together with respect to the other parts of the support device such that the at least two support rolls of the first group stay in contact with the patient bearing device during the bending.

35. The patient positioning system according to claim 22, wherein the at least two support rolls of the first group can be pivoted together only in a pivot range of 20 degrees or less with respect to the other parts of the support device.

36. A medical table, comprising a column, and a patient positioning system according to claim 22, the patient positioning system being fixedly or detachably connected to the column.

37. A patient positioning system usable as a part of a medical table, the patient positioning system comprising:
a patient bearing device configured to bear a patient;
a pair of support rolls on which the patient bearing device lies, the patient bearing device being movable with respect to the pair of support rolls to thereby facilitate movement of a patient, and each of the support rolls of the pair of support rolls being independently rotatable about its respective rotational axis; and
a height adjustment element configured to adjust a height of the pair of support rolls.

38. The patient positioning system according to claim 37, wherein the height adjustment element comprises:
first and second bearing blocks, the first bearing block bearing first ends of the pair of support rolls and the second bearing block bearing second ends of the pair of support rolls; and
first and second threaded elements, the first threaded element configured to adjust the height of the first bearing block, and the second threaded element configured to adjust the height of the second bearing block.

39. The patient positioning system according to claim 38, further comprising first and second support blocks configured to pivotably support the first and second bearing blocks, respectively, the first and second support blocks each have a support section on which a respective receiving section of the corresponding first or second bearing block is supported, each of the support sections of the first and second support blocks having a shape that is complementary to a shape of the corresponding receiving section of the corresponding first or second bearing block.

40. The patient positioning system according to claim 39, wherein the first and second support blocks each have a bore configured to receive a corresponding one of the first or second threaded element, the first or second threaded element configured to adjustably connect the corresponding first or second support block to a base element and to adjust the height of its corresponding first or second bearing block.

41. A medical table, comprising a column, and a patient positioning system according to claim 40, the patient positioning system being fixedly or detachably connected to the column.
